(19) United States
(12) Patent Application Publication
(10) Pub. No.: US 2023/0035954 A1
(43) Pub. Date: Feb. 2, 2023

(54) METHOD FOR ESTABLISHING MEDICINE SYNERGISM PREDICTION MODEL, PREDICTION METHOD AND CORRESPONDING APPARATUS

(71) Applicant: BEIJING BAIDU NETCOM SCIENCE TECHNOLOGY CO., LTD., Beijing (CN)

(72) Inventors: Jing HU, Beijing (CN); Guodong ZHAO, Beijing (CN)

(73) Assignee: BEIJING BAIDU NETCOM SCIENCE TECHNOLOGY CO., LTD., Beijing (CN)

(21) Appl. No.: 17/844,094

(22) Filed: Jun. 20, 2022

(30) Foreign Application Priority Data
Jul. 29, 2021 (CN) 202110867932.6
Dec. 24, 2021 (CN) 202111597730.0

(51) Int. Cl. G16B 45/00; G16B 40/20; G16H 50/50; G06K 9/62; G06F 16/901

(52) U.S. Cl. CPC G16B 45/00; G06F 16/9024; G06K 9/6256; G16B 40/20; G16H 50/50

(57) ABSTRACT

The present disclosure discloses a method for establishing a medicine synergism prediction model, a prediction method and corresponding apparatus, and relates to deep learning and artificial intelligence (AI) medical technologies in the field of AI technologies. A specific implementation solution includes: acquiring a relation graph, nodes in the relation graph including medicine nodes and protein nodes, and edges indicating that interaction exists between the nodes; collecting, from the relation graph, a medicine node pair with definite synergism and a label of whether the medicine node pair has synergism as training samples; and training the medicine synergism prediction model by taking the medicine node pair in the training samples as input to the medicine synergism prediction model and taking the label of whether the medicine node pair has synergism as target output; wherein the medicine synergism prediction model is obtained by learning the relation graph based on a graph convolutional network.

### CROSS-REFERENCE TO RELATED APPLICATIONS

The present application claims the priority of Chinese Patent Application No. 202110867932.6, filed on Jul. 29, 2021, with the title of “METHOD FOR ESTABLISHING MEDICINE SYNERGISM PREDICTION MODEL, PREDICTION METHOD AND CORRESPONDING APPARATUS” and the priority of Chinese Patent Application No. 202111597730.0, filed on Dec. 24, 2021, with the title of “METHOD FOR ESTABLISHING MEDICINE SYNERGISM PREDICTION MODEL, PREDICTION METHOD AND CORRESPONDING APPARATUS.” The disclosures of the above applications are incorporated herein by reference in their entirety.

### FIELD OF THE DISCLOSURE

The present disclosure relates to the field of computer application technologies, and in particular, to deep learning and artificial intelligence (AI) medical technologies in the field of AI technologies.

### BACKGROUND OF THE DISCLOSURE

Combination medication refers to simultaneous or sequential application of two or more medicines for the purpose of treatment, mainly to increase the efficacy of the medicines or reduce toxic and side effects of the medicines. However, opposite results may also be produced. Therefore, rational Combination medication is very important. The rational Combination medication is based on medicine synergism. However, screening the medicine synergism from the experimental end consumes lots of manpower and material resources.

### SUMMARY OF THE DISCLOSURE

In view of the above, the present disclosure provides a method for establishing a medicine synergism prediction model, a prediction method and corresponding apparatus, so as to reduce labor and material costs.

According to a first aspect of the present disclosure, a method for establishing a medicine synergism prediction model is provided, including acquiring a relation graph, nodes in the relation graph including medicine nodes and protein nodes, and edges indicating that interaction exists between the nodes; collecting, from the relation graph, a medicine node pair with definite synergism and a label of whether the medicine node pair has synergism as training samples; and training the medicine synergism prediction model by taking the medicine node pair in the training samples as input to the medicine synergism prediction model and taking the label of whether the medicine node pair has synergism as target output; wherein the medicine synergism prediction model is obtained by learning the relation graph based on a graph convolutional network.

According to a second aspect of the present disclosure, a medicine synergism prediction method is provided, including determining a to-be-identified medicine node pair from a relation graph; and predicting the to-be-identified medicine node pair by using a medicine synergism prediction model, to obtain a prediction result indicating whether the to-be-identified medicine node pair has synergism; wherein the medicine synergism prediction model is pre-trained with the method as described above.

According to a third aspect of the present disclosure, an electronic device, including at least one processor; and a memory communicatively connected with the at least one processor; wherein the memory stores instructions executable by the at least one processor, and the instructions are executed by the at least one processor to enable the at least one processor to perform a method for establishing a medicine synergism prediction model, wherein the method includes acquiring a relation graph, nodes in the relation graph including medicine nodes and protein nodes, and edges indicating that interaction exists between the nodes; collecting, from the relation graph, a medicine node pair with definite synergism and a label of whether the medicine node pair has synergism as training samples; and training the medicine synergism prediction model by taking the medicine node pair in the training samples as input to the medicine synergism prediction model and taking the label of whether the medicine node pair has synergism as target output; wherein the medicine synergism prediction model is obtained by learning the relation graph based on a graph convolutional network.

According to a fourth aspect of the present disclosure, there is provided a non-transitory computer readable storage medium with computer instructions stored thereon, wherein the computer instructions are used for causing a computer to perform a method for establishing a medicine synergism prediction model, wherein the method includes acquiring a relation graph, nodes in the relation graph including medicine nodes and protein nodes, and edges indicating that interaction exists between the nodes; collecting, from the relation graph, a medicine node pair with definite synergism and a label of whether the medicine node pair has synergism as training samples; and training the medicine synergism prediction model by taking the medicine node pair in the training samples as input to the medicine synergism prediction model and taking the label of whether the medicine node pair has synergism as target output; wherein the medicine synergism prediction model is obtained by learning the relation graph based on a graph convolutional network.

It should be understood that the content described in this part is neither intended to identify key or significant features of the embodiments of the present disclosure, nor intended to limit the scope of the present disclosure. Other features of the present disclosure will be made easier to understand through the following description.

### BRIEF DESCRIPTION OF DRAWINGS

The accompanying drawings are intended to provide a better understanding of the solutions and do not constitute a limitation on the present disclosure. In the drawings,

FIG. 1 is a flowchart of a method for establishing a medicine synergism prediction model according to an embodiment of the present disclosure;

FIG. 2 is a schematic relation graph according to an embodiment of the present disclosure;

FIG. 3 is a schematic diagram of the medicine synergism prediction model according to an embodiment of the present disclosure;

FIG. 4 is a flowchart of a medicine synergism prediction method according to an embodiment of the present disclosure;

FIG. 5 is a structural diagram of an apparatus for establishing a medicine synergism prediction model according to an embodiment of the present disclosure;

FIG. 6 is a structural diagram of a medicine synergism prediction apparatus according to an embodiment of the present disclosure; and

FIG. 7 is a block diagram of an electronic device configured to implement embodiments of the present disclosure.

### DETAILED DESCRIPTION OF PREFERRED EMBODIMENTS

Exemplary embodiments of the present disclosure are illustrated below with reference to the accompanying drawings, which include various details of the present disclosure to facilitate understanding and should be considered only as exemplary. Therefore, those of ordinary skill in the art should be aware that various changes and modifications can be made to the embodiments described herein without departing from the scope and spirit of the present disclosure. Similarly, for clarity and simplicity, descriptions of well-known functions and structures are omitted in the following description.

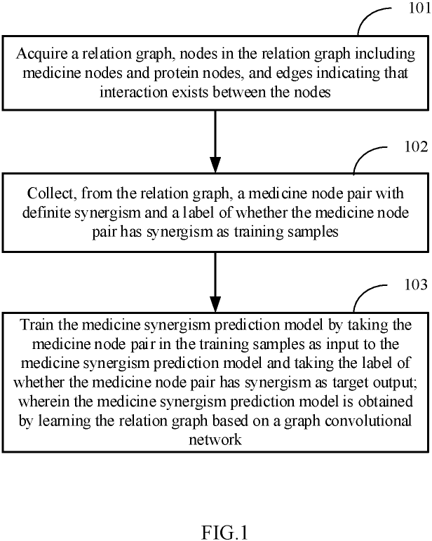

The present disclosure provides a manner of establishing a medicine synergism prediction model based on a graph convolutional network and a prediction manner based on the model. FIG. 1 is a flowchart of a method for establishing a medicine synergism prediction model according to an embodiment of the present disclosure. The method is performed by an apparatus for establishing a medicine synergism prediction model. The apparatus may be an application located in a computer terminal or a functional unit in an application located in a computer terminal such as a plug-in or a Software Development Kit (SDK), or located on a server side, which is not particularly limited herein in the embodiment of the present invention. As shown in FIG. 1, the method may include the following steps.

In 101, a relation graph is acquired, nodes in the relation graph including medicine nodes and protein nodes, and edges indicating that interaction exists between the nodes.

In 102, a medicine node pair with definite synergism and a label of whether the medicine node pair has synergism are collected from the relation graph as training samples.

In 103, the medicine synergism prediction model is trained by taking the medicine node pair in the training samples as input to the medicine synergism prediction model and taking the label of whether the medicine node pair has synergism as target output; wherein the medicine synergism prediction model is obtained by learning the relation graph based on a graph convolutional network.

As can be seen, in the present disclosure, the medicine synergism prediction model is obtained by learning interaction between the medicine node pair with definite synergism in the relation graph in a preset neighborhood range based on the graph convolutional network. Based on this, automatic prediction of medicine synergism can be realized, which saves more labor and material costs than all trials.

The above steps are described in detail below with reference to embodiments. Firstly, step 101 is described in detail.

Medicines mainly act on animals (including humans), and distribution, transport, metabolism and efficacy of the medicines in the animals are all related to protein. Therefore, the study on medicine effects mainly focuses on the interaction between medicines and protein, and the protein on which the medicines act are generally called target protein. That is, the interaction between the medicines and the protein may be acquired from previous experimental data.

The interaction between the protein is also a relatively mature technology, which has accumulated a large number of experimental data and is in the process of continuous development. The interaction between the protein can better annotate protein functions and decode life phenomena, and is particularly useful in medicine design.

Part of medicine node pairs with definite synergism have been obtained through experiments in the early stage, and this part of experimental data can also be acquired and used for model training in the present disclosure.

A relation graph may be constructed by using data of the interaction between medicines and protein, between protein and between medicines. The relation graph consists of nodes and edges. The nodes include medicines and protein. Edges between the nodes indicate that interaction exists between the nodes. The interaction between the medicines includes synergism. That is, if synergism exists between the medicines, edges exist between corresponding medicine nodes in the relation graph. Otherwise, no edges exist.

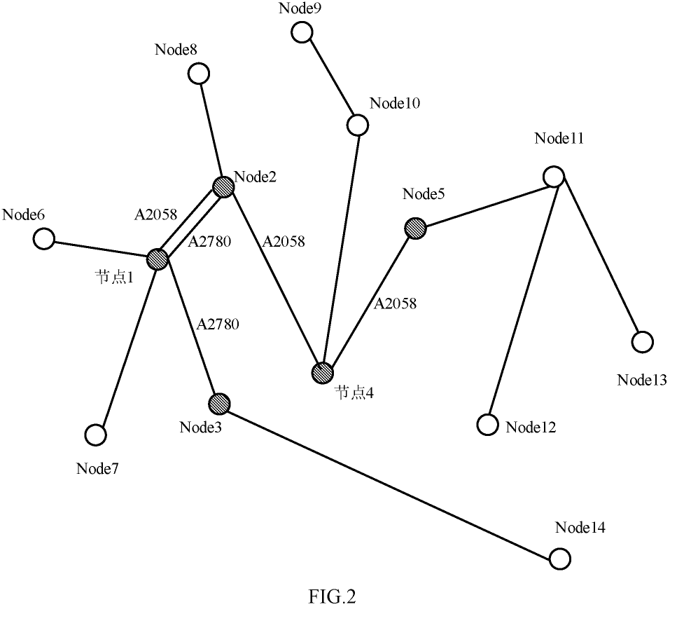

FIG. 2 is a schematic relation graph according to an embodiment of the present disclosure. In the figure, solid nodes 1-5 represent medicine nodes, and hollow nodes 6-14 represent protein nodes. Edges between the medicine nodes and the protein nodes represent interaction between medicines and protein, and edges between the protein nodes represent interaction between the corresponding protein. Edges between the medicine nodes indicate that synergism exists between the medicine nodes.

Furthermore, since the synergism between the medicine nodes vary with different cell lines, the cell lines are mainly configured to distinguish sites or lesions. Cancer, for example, is generally differentiated by primary lesions. Therefore, as a preferred implementation, in the relation graph shown in FIG. 2, edges between the medicine nodes are identified by cell lines. For example, synergism exists between Node 1 and Node 2 in cell lines A2058 and A2780. Therefore, edges identified by A2058 and A2780 respectively exist between Node 1 and Node 2. In addition to the identification manner, nodes corresponding to edges of different cell lines may also be regarded as different nodes. For example, Node 1 combined with A2058 is regarded as one node, and Node 1 combined with A2780 is regarded as another node.

In the relation graph, edge connections exist between the medicine node pairs with definite synergism, and no edge exists between some medicine nodes with indefinite synergism or definitely without synergism. In step 102 of the above embodiment, a medicine node pair with definite synergism and a label of whether the medicine node pair has synergism may be collected from the relation graph as training samples. Furthermore, since synergism between the medicine node pairs, i.e., edges between the medicine nodes, is identified by cell lines in the relation graph, the label of the training sample includes cell line tags.

Taking FIG. 2 as an example, definite synergism exists between Medicine Node 1 and Medicine Node 2. Therefore, Medicine Node 1 and Medicine Node 2 may be collected as a medicine node pair to form a training sample, for example,
training sample 1: (Medicine Node 1-Medicine Node 2, having synergism [A2058]);
training sample 2: (Medicine Node 1-Medicine Node 2, having synergism [A2780]).
   Synergism definitely does not exist between Medicine Node 2 and Medicine Node 5. Therefore, Medicine Node 2 and Medicine Node 5 may be collected as a medicine node pair to form a training sample, for example,
training sample 3: (Medicine Node 2-Medicine Node 5, having no synergism).

Step 103 “training the medicine synergism prediction model by taking the medicine node pair in the training samples as input to the medicine synergism prediction model and taking the label of whether the medicine node pair has synergism as target output” is described in detail below with reference to embodiments.

In the present disclosure, during the training of the medicine synergism prediction model, connection relations between the medicine nodes in the training samples reflected in the relation graph are learned based on a graph convolutional network. In addition to relations between the medicine nodes, the connection relations further include relations between the medicine nodes and the protein as well as relations between the protein. However, if the relation graph is fully learned, an adjacent matrix is huge and the training efficiency is low. Therefore, in the embodiment of the present disclosure, for the medicine nodes, nodes and edges are selected within a preset neighborhood range to form a subgraph, and the subgraph is learned, so as to improve the learning efficiency.

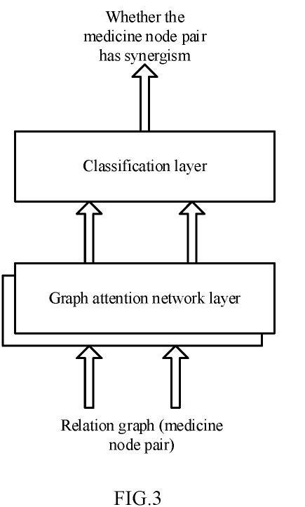

Specifically, as shown in FIG. 3, the medicine synergism prediction model may mainly include a graph convolutional network layer and a classification layer. The graph convolutional network layer may include more than one graph convolutional network layer.

After the medicine nodes in the training samples are inputted to the medicine synergism prediction model, the graph convolutional network layer is configured to acquire, from the relation graph, subgraphs corresponding to the medicine nodes in the inputted medicine node pair. The subgraphs corresponding to the medicine nodes include medicine nodes, neighbor nodes of the medicine nodes within a preset neighborhood range and edges between the medicine nodes and the neighbor nodes. Then, graph convolution processing is performed on the subgraphs corresponding to the medicine nodes to obtain vector representations corresponding to the medicine nodes.

The preset neighborhood range may include a first-order protein neighbor node and neighbor protein nodes of the first-order protein neighbor node. That is, during sampling learning, the first-order neighbor node of the medicine node, namely, target protein of the medicine node, is sampled, and then a neighbor node of the first-order neighbor node (namely, a second-order neighbor of the medicine node) is sampled. However, the sampling of the second-order neighbor nodes does not include nodes that have been sampled. In the above process, the sampling of the first-order neighbor is actually sampling a medicine-target relation, and the sampling of the second-order neighbor is actually sampling a protein-protein relation.

When graph convolution processing is performed on the subgraphs corresponding to the medicine nodes, features of the nodes and features of the edges included in the subgraphs corresponding to the medicine nodes may be embedded. Then, feature vectors of the nodes and feature vectors of the edges obtained by embedding are aggregated, to obtain the vector representations corresponding to the medicine nodes.

Taking a medicine node pair consisting of Node 1 and Node 2 in FIG. 2 as an example, first-order neighbors of Node 1, namely Node 6 and Node 7, are sampled. A second-order neighbor node of Node 1, namely Node 14, is sampled. Graph convolution processing is performed on neighbor nodes and edges within the neighborhood range, including embedding features of Nodes 1, 6 and 7, embedding features of an edge between Node 1 and Node 6 and an edge between Node 1 and Node 7 and an edge between Node 7 and Node 14, and then aggregating feature vectors obtained by embedding, to obtain a vector representation corresponding to Node 1.

A first-order neighbor node of Node 2, namely Node 8, is sampled, and a second-order neighbor node of Node 2, namely Node 9, is sampled Graph convolution processing is performed on neighbor nodes and edges within the neighborhood range, including embedding features of Nodes 2, 8 and 9, embedding features of an edge between Node 2 and Node 8 and an edge between Node 8 and Node 9, and then aggregating feature vectors obtained by embedding, to obtain a vector representation corresponding to Node 2.

During the above embedding, the features of the edges used may include a type of interaction, a cell line, a gene expression profile and other transcriptome information. Initial values used during the embedding may be either preset values or pre-trained by a disease classification task. For example, a cancer-type classification task may be used. For example, a cancer-type classification model is used. The classification model includes a first embedding unit and a mapping unit. The first embedding unit embeds features of transcriptome, and the mapping unit maps results obtained by embedding to specific cancer types. During the training, transcriptome corresponding to known cancer types is used as training data to train the above cancer-type classification model. That is, the transcriptome is used as input and the corresponding known cancer type is used as target output. After the training, the first embedding unit in the trained cancer-type classification model is used as a pre-training model, edges obtained by sampling in the present disclosure are embedded, and the obtained feature vectors are used as initial values of the feature vectors of the edges to train the medicine synergism prediction model.

The features of the nodes used may include molecular weight, molecular activity, etc. In the embodiment of the present disclosure, initial values used by the medicine synergism prediction model during the embedding may be either preset values or pre-trained by a compound-compound interaction (CCI) task. For example, results of interaction between compound pairs are collected as training samples, the compound pairs are taken as input, and the corresponding results of interaction are taken as target output to train the CCI classification model. The CCI classification model includes a second embedding unit and a mapping unit. The second embedding unit embeds features of compounds in the compound pairs, and the mapping unit maps results obtained by embedding to specific interaction results. After the training, the trained second embedding unit is used as a pre-training model, nodes used in the present disclosure are embedded, and the obtained feature vectors are used as initial values of the feature vectors of the nodes to train the medicine synergism prediction model.

Still refer to FIG. 3. The classification layer is configured to obtain, by using the vector representations of the medicine nodes in the medicine node pair, a classification result indicating whether the medicine node pair has synergism. A training objective of the medicine synergism prediction model is to minimize a difference between the classification result and the corresponding label.

Still taking FIG. 2 as an example, after the vector representation of Node 1 and the vector representation of Node 2 are obtained in the above process, the classification layer performs classification by using the vector representation corresponding to Node 1 and the vector representation corresponding to Node 2. The classification result is whether the medicine node pair has synergism. In an actual classification process, a probability value of existence of synergism between Node 1 and Node 2 is actually obtained.

Furthermore, during the above learning, since the edges are distinguished by different cell lines, cell line information is included in the classification result. That is, the obtained classification result is actually whether a medicine pair has synergism on a cell line.

As can be seen, the medicine synergism prediction model established above not only retains interaction characteristics of biological networks, but also introduces the characterization of different cell lines, so that the established model has better discrimination capability.

During the above training, a loss function may be constructed by using the training objective. In each iteration, model parameters are updated with the value of the loss function until a preset training end condition is reached. The training end condition may be such as convergence of the loss function or a number of iterations reaching a preset number threshold.

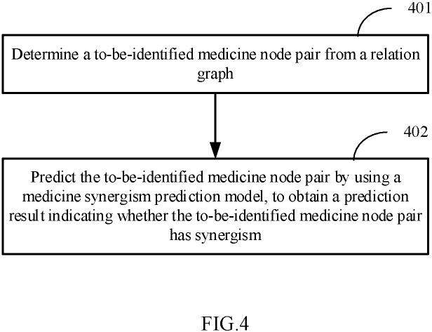

FIG. 4 is a flowchart of a medicine synergism prediction method according to an embodiment of the present disclosure. The method is performed by a medicine synergism prediction apparatus. The apparatus may be an application located in a computer terminal or a functional unit in an application located in a computer terminal such as a plug-in or an SDK, or located on a server side, which is not particularly limited herein in the embodiment of the present invention. As shown in FIG. 4, the method may include the following steps.

In 401, a to-be-identified medicine node pair is determined from a relation graph.

In the embodiment of the present disclosure, a medicine node pair with indefinite synergism in the relation graph may be predicted. That is, the medicine node pair with indefinite synergism in the relation graph may be taken as the to-be-identified medicine node pair.

For a newly generated medicine, the interaction between the new medicine and protein has to be verified by experiments, so it may be acquired through experimental data. In this case, the new medicine may be added to the relation graph to predict synergism with other medicine nodes. That is, the new medicine and other medicine nodes in the relation graph may form to-be-identified medicine node pairs respectively.

In 402, the to-be-identified medicine node pair is predicted by using a medicine synergism prediction model, to obtain a prediction result indicating whether the to-be-identified medicine node pair has synergism.

After the to-be-identified medicine node pair is inputted to the medicine synergism prediction model, the graph convolutional network layer of the medicine synergism prediction model acquires subgraphs corresponding to the medicine nodes in the inputted medicine node pair, the subgraphs corresponding to the medicine nodes including medicine nodes, neighbor nodes of the medicine nodes within a preset neighborhood range and edges between the medicine nodes and the neighbor nodes; and performs graph convolution processing on the subgraphs corresponding to the medicine nodes to obtain vector representations corresponding to the medicine nodes. Then, the classification layer obtains, by using the vector representations of the medicine nodes in the medicine node pair, a classification result indicating whether the medicine node pair has synergism. The classification result is a prediction result.

Furthermore, since an identifier of a cell line is incorporated into the edge feature of the medicine synergism prediction model, during the above prediction, a target cell line may be further determined in step 401. That is, synergism of the medicine pair on a specific cell line is predicted. The medicine synergism prediction model considers a relation between edges of nodes in the target cell line, to obtain a prediction result indicating whether the to-be-identified medicine node pair has synergism in the target cell line.

The above is a detailed description of the method according to the present disclosure, and the following is a detailed description of the apparatus according to the present disclosure with reference to embodiments.

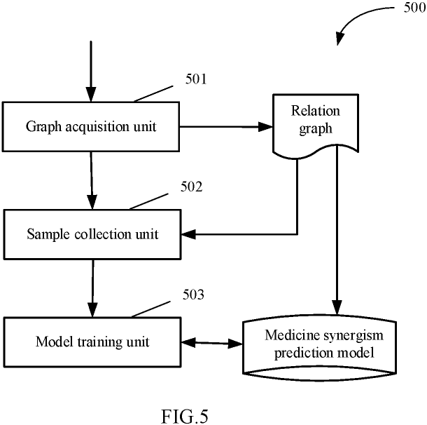

FIG. 5 is a structural diagram of an apparatus for establishing a medicine synergism prediction model according to an embodiment of the present disclosure. As shown in FIG. 5, the apparatus 500 may include: a graph acquisition unit 501, a sample collection unit 502 and a model training unit 503. Main functions of the component units are as follows.

The graph acquisition unit 501 is configured to acquire a relation graph, nodes in the relation graph including medicine nodes and protein nodes, and edges indicating that interaction exists between the nodes.

The sample collection unit 502 is configured to collect, from the relation graph, a medicine node pair with definite synergism and a label of whether the medicine node pair has synergism as training samples.

The model training unit 503 is configured to train the medicine synergism prediction model by taking the medicine node pair in the training samples as input to the medicine synergism prediction model and taking the label of whether the medicine node pair has synergism as target output; wherein the medicine synergism prediction model is obtained by learning the relation graph based on a graph convolutional network.

The medicine synergism prediction model may include a graph convolutional network layer and a classification layer.

The graph convolutional network layer is configured to acquire, from the relation graph, subgraphs corresponding to the medicine nodes in the inputted medicine node pair, the subgraphs corresponding to the medicine nodes including medicine nodes, neighbor nodes of the medicine nodes within a preset neighborhood range and edges between the medicine nodes and the neighbor nodes; and performing graph convolution processing on the subgraphs corresponding to the medicine nodes to obtain vector representations corresponding to the medicine nodes.

The classification layer is configured to obtain, by using the vector representations of the medicine nodes in the medicine node pair, a classification result indicating whether the medicine node pair has synergism.

A training objective of the model training unit 503 is to minimize a difference between the classification result and the corresponding label.

As an implementation, the preset neighborhood range includes: a first-order protein neighbor node and neighbor protein nodes of the first-order protein neighbor node.

Furthermore, since the synergism between the medicine nodes vary with different cell lines, as a preferred implementation, edges between the medicine nodes are identified by cell lines in the relation graph, and the label of the training sample includes cell line tags. Correspondingly, the classification result is whether a medicine pair has synergism in the cell lines.

As an implementation, when performing graph convolution processing on the subgraphs corresponding to the medicine nodes, the graph convolutional network layer is specifically configured to: embed features of the nodes and features of the edges included in the subgraphs corresponding to the medicine nodes; and aggregate feature vectors of the nodes and feature vectors of the edges obtained by embedding, to obtain the vector representations corresponding to the medicine nodes.

Initial values of the feature vectors of the edges are pre-trained by a disease classification task. Initial values of the feature vectors of the nodes are pre-obtained by a CCI task.

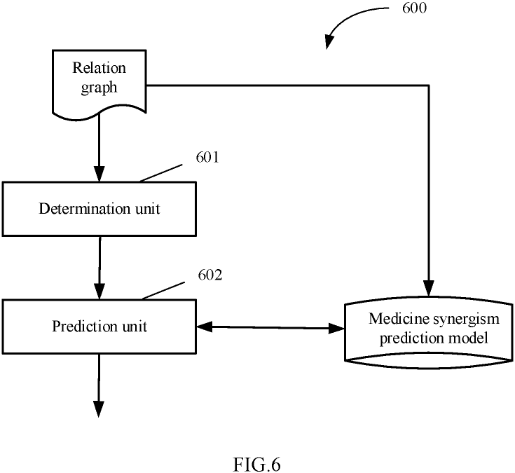

FIG. 6 is a structural diagram of a medicine synergism prediction apparatus according to an embodiment of the present disclosure. As shown in FIG. 6, the apparatus 600 may include: a determination unit 601 and a prediction unit 602. Main functions of the component units are as follows.

The determination unit 601 is configured to determine a to-be-identified medicine node pair from a relation graph.

The prediction unit 602 is configured to predict the to-be-identified medicine node pair by using a medicine synergism prediction model, to obtain a prediction result indicating whether the to-be-identified medicine node pair has synergism.

The medicine synergism prediction model is pre-trained by the apparatus shown in FIG. 5.

As a preferred implementation, the determination unit 601 may further determine a target cell line. Correspondingly, the prediction unit 602 obtains the prediction result indicating whether the to-be-identified medicine node pair has synergism in the target cell line.

Various embodiments in the specification are described progressively. Same and similar parts among the embodiments may be referred to one another, and each embodiment focuses on differences from other embodiments. In particular, the apparatus embodiments are basically similar to the method embodiments, so the description thereof is relatively simple. Related parts may be obtained with reference to the corresponding description in the method embodiments.

Acquisition, storage and application of users' personal information involved in the technical solutions of the present disclosure comply with relevant laws and regulations, and do not violate public order and moral.

According to embodiments of the present disclosure, the present application further provides an electronic device, a readable storage medium and a computer program product.

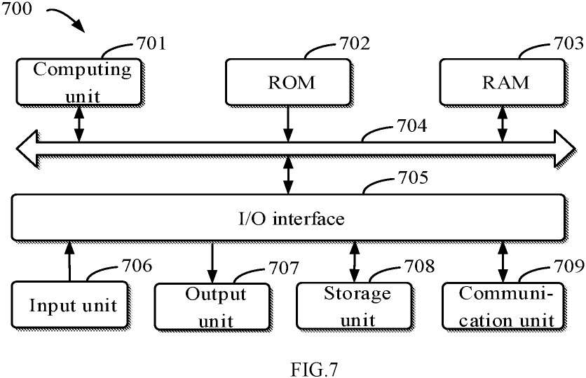

FIG. 7 is a block diagram of an electronic device configured to perform a method for establishing a medicine synergism prediction model and a prediction method according to embodiments of the present disclosure. The electronic device is intended to represent various forms of digital computers, such as laptops, desktops, workbenches, personal digital assistants, servers, blade servers, mainframe computers and other suitable computing devices. The electronic device may further represent various forms of mobile devices, such as personal digital assistants, cellular phones, smart phones, wearable devices and other similar computing devices. The components, their connections and relationships, and their functions shown herein are examples only, and are not intended to limit the implementation of the present disclosure as described and/or required herein.

As shown in FIG. 7, the device 700 includes a computing unit 701, which may perform various suitable actions and processing according to a computer program stored in a read-only memory (ROM) 702 or a computer program loaded from a storage unit 708 into a random access memory (RAM) 703. The RAM 703 may also store various programs and data required to operate the device 700. The computing unit 701, the ROM 702 and the RAM 703 are connected to one another by a bus 704. An input/output (I/O) interface 705 may also be connected to the bus 704.

A plurality of components in the device 700 are connected to the I/O interface 705, including an input unit 706, such as a keyboard and a mouse; an output unit 707, such as various displays and speakers; a storage unit 708, such as disks and discs; and a communication unit 709, such as a network card, a modem and a wireless communication transceiver. The communication unit 709 allows the device 700 to exchange information/data with other devices over computer networks such as the Internet and/or various telecommunications networks.

The computing unit 701 may be a variety of general-purpose and/or special-purpose processing components with processing and computing capabilities. Some examples of the computing unit 701 include, but are not limited to, a central processing unit (CPU), a graphics processing unit (GPU), various artificial intelligence (AI) computing chips, various computing units that run machine learning model algorithms, a digital signal processor (DSP), and any appropriate processor, controller or microcontroller, etc. The computing unit 701 performs the methods and processing described above, such as the method for establishing a medicine synergism prediction model and the prediction method. For example, in some embodiments, the method for establishing a medicine synergism prediction model and the prediction method may be implemented as a computer software program that is tangibly embodied in a machine-readable medium, such as the storage unit 708.

In some embodiments, part or all of a computer program may be loaded and/or installed on the device 700 via the ROM 702 and/or the communication unit 709. One or more steps of the method for establishing a medicine synergism prediction model and the prediction method described above may be performed when the computer program is loaded into the RAM 703 and executed by the computing unit 701. Alternatively, in other embodiments, the computing unit 701 may be configured to perform the method for establishing a medicine synergism prediction model and the prediction method by any other appropriate means (for example, by means of firmware).

Various implementations of the systems and technologies disclosed herein can be realized in a digital electronic circuit system, an integrated circuit system, a field programmable gate array (FPGA), an application-specific integrated circuit (ASIC), an application-specific standard product (ASSP), a system on chip (SOC), a complex programmable logic device (CPLD), computer hardware, firmware, software, and/or combinations thereof. Such implementations may include implementation in one or more computer programs that are executable and/or interpretable on a programmable system including at least one programmable processor, which can be special or general purpose, configured to receive data and instructions from a storage system, at least one input apparatus, and at least one output apparatus, and to transmit data and instructions to the storage system, the at least one input apparatus, and the at least one output apparatus.

Program codes configured to implement the methods in the present disclosure may be written in any combination of one or more programming languages. Such program codes may be supplied to a processor or controller of a general-purpose computer, a special-purpose computer, or another programmable data processing apparatus to enable the function/operation specified in the flowchart and/or block diagram to be implemented when the program codes are executed by the processor or controller. The program codes may be executed entirely on a machine, partially on a machine, partially on a machine and partially on a remote machine as a stand-alone package, or entirely on a remote machine or a server.

In the context of the present disclosure, machine-readable media may be tangible media which may include or store programs for use by or in conjunction with an instruction execution system, apparatus or device. The machine-readable media may be machine-readable signal media or machine-readable storage media. The machine-readable media may include, but are not limited to, electronic, magnetic, optical, electromagnetic, infrared, or semiconductor systems, apparatus or devices, or any suitable combinations thereof. More specific examples of machine-readable storage media may include electrical connections based on one or more wires, a portable computer disk, a hard disk, an RAM, an ROM, an erasable programmable read only memory (EPROM or flash memory), an optical fiber, a compact disk read only memory (CD-ROM), an optical storage device, a magnetic storage device, or any suitable combination thereof.

To provide interaction with a user, the systems and technologies described here can be implemented on a computer. The computer has: a display apparatus (e.g., a cathode-ray tube (CRT) or a liquid crystal display (LCD) monitor) for displaying information to the user; and a keyboard and a pointing apparatus (e.g., a mouse or trackball) through which the user may provide input for the computer. Other kinds of apparatus may also be configured to provide interaction with the user. For example, a feedback provided for the user may be any form of sensory feedback (e.g., visual, auditory, or tactile feedback); and input from the user may be received in any form (including sound input, voice input, or tactile input).

The systems and technologies described herein can be implemented in a computing system including background components (e.g., as a data server), or a computing system including middleware components (e.g., an application server), or a computing system including front-end components (e.g., a user computer with a graphical user interface or web browser through which the user can interact with the implementation schema of the systems and technologies described here), or a computing system including any combination of such background components, middleware components or front-end components. The components of the system can be connected to each other through any form or medium of digital data communication (e.g., a communication network). Examples of the communication network include: a local area network (LAN), a wide area network (WAN) and the Internet.

The computer system may include a client and a server. The client and the server are generally far away from each other and generally interact via the communication network. A relationship between the client and the server is generated through computer programs that run on a corresponding computer and have a client-server relationship with each other. The server may be a cloud server, also known as a cloud computing server or cloud host, which is a host product in the cloud computing service system to solve the problems of difficult management and weak business scalability in the traditional physical host and a virtual private server (VPS). The server may also be a distributed system server, or a server combined with blockchain.

It should be understood that the steps can be reordered, added, or deleted using the various forms of processes shown above. For example, the steps described in the present application may be executed in parallel or sequentially or in different sequences, provided that desired results of the technical solutions disclosed in the present disclosure are achieved, which is not limited herein.

The above specific implementations do not limit the protection scope of the present disclosure. Those skilled in the art should understand that various modifications, combinations, sub-combinations, and replacements can be made according to design requirements and other factors. Any modifications, equivalent substitutions and improvements made within the spirit and principle of the present disclosure all should be included in the protection scope of the present disclosure.

What is claimed is:

1. A method for establishing a medicine synergism prediction model, comprising:
acquiring a relation graph, nodes in the relation graph comprising medicine nodes and protein nodes, and edges indicating that interaction exists between the nodes;
collecting, from the relation graph, a medicine node pair with definite synergism and a label of whether the medicine node pair has synergism as training samples; and
training the medicine synergism prediction model by taking the medicine node pair in the training samples as input to the medicine synergism prediction model and taking the label of whether the medicine node pair has synergism as target output; wherein the medicine synergism prediction model is obtained by learning the relation graph based on a graph convolutional network.

2. The method according to claim 1, wherein the medicine synergism prediction model is obtained by learning the subgraph formed by some nodes and edges in the relation graph based on the graph convolutional network.

3. The method according to claim 1, wherein the medicine synergism prediction model comprises a graph convolutional network layer and a classification layer;
the graph convolutional network layer is configured to acquire, from the relation graph, subgraphs corresponding to the medicine nodes in the inputted medicine node pair, the subgraphs corresponding to the medicine nodes comprising medicine nodes, neighbor nodes of the medicine nodes within a preset neighborhood range and edges between the medicine nodes and the neighbor nodes; and performing graph convolution processing on the subgraphs corresponding to the medicine nodes to obtain vector representations corresponding to the medicine nodes;
the classification layer is configured to obtain, by using the vector representations of the medicine nodes in the medicine node pair, a classification result indicating whether the medicine node pair has synergism; and
a training objective of the medicine synergism prediction model is to minimize a difference between the classification result and the corresponding label.

4. The method according to claim 3, wherein the preset neighborhood range comprises: a first-order protein neighbor node and neighbor protein nodes of the first-order protein neighbor node.

5. The method according to claim 3, wherein edges between the medicine nodes in the relation graph are identified by cell lines, and the label of the training sample comprises cell line tags; and
the classification result is whether a medicine pair has synergism in the cell lines.

6. The method according to claim 3, wherein the step of performing graph convolution processing on the subgraphs corresponding to the medicine nodes comprises:
embedding features of the nodes and features of the edges comprised in the subgraphs corresponding to the medicine nodes; and
aggregating feature vectors of the nodes and feature vectors of the edges obtained by embedding, to obtain the vector representations corresponding to the medicine nodes.

7. The method according to claim 6, wherein initial values of the feature vectors of the edges are pre-trained by a disease classification task; and
initial values of the feature vectors of the nodes are pre-obtained by a compoundcompound interaction (CCI) task.

8. The method according to claim 2, wherein the medicine synergism prediction model comprises a graph convolutional network layer and a classification layer;
the graph convolutional network layer is configured to acquire, from the relation graph, subgraphs corresponding to the medicine nodes in the inputted medicine node pair, the subgraphs corresponding to the medicine nodes comprising medicine nodes, neighbor nodes of the medicine nodes within a preset neighborhood range and edges between the medicine nodes and the neighbor nodes; and performing graph convolution processing on the subgraphs corresponding to the medicine nodes to obtain vector representations corresponding to the medicine nodes;
the classification layer is configured to obtain, by using the vector representations of the medicine nodes in the medicine node pair, a classification result indicating whether the medicine node pair has synergism; and
a training objective of the medicine synergism prediction model is to minimize a difference between the classification result and the corresponding label.

9. A medicine synergism prediction method, comprising:
determining a to-be-identified medicine node pair from a relation graph; and
predicting the to-be-identified medicine node pair by using a medicine synergism prediction model, to obtain a prediction result indicating whether the to-be-identified medicine node pair has synergism;
wherein the medicine synergism prediction model is pre-trained with the method according to claim 1.

10. An electronic device, comprising:
at least one processor; and
a memory communicatively connected with the at least one processor;
wherein the memory stores instructions executable by the at least one processor, and the instructions are executed by the at least one processor to enable the at least one processor to perform a method for establishing a medicine synergism prediction model, wherein the method comprises:
acquiring a relation graph, nodes in the relation graph comprising medicine nodes and protein nodes, and edges indicating that interaction exists between the nodes;
collecting, from the relation graph, a medicine node pair with definite synergism and a label of whether the medicine node pair has synergism as training samples; and
training the medicine synergism prediction model by taking the medicine node pair in the training samples as input to the medicine synergism prediction model and taking the label of whether the medicine node pair has synergism as target output; wherein the medicine synergism prediction model is obtained by learning the relation graph based on a graph convolutional network.

11. The electronic device according to claim 10, wherein the medicine synergism prediction model is obtained by learning the subgraph formed by some nodes and edges in the relation graph based on the graph convolutional network.

12. The electronic device according to claim 10, wherein the medicine synergism prediction model comprises a graph convolutional network layer and a classification layer;
the graph convolutional network layer is configured to acquire, from the relation graph, subgraphs corresponding to the medicine nodes in the inputted medicine node pair, the subgraphs corresponding to the medicine nodes comprising medicine nodes, neighbor nodes of the medicine nodes within a preset neighborhood range and edges between the medicine nodes and the neighbor nodes; and performing graph convolution processing on the subgraphs corresponding to the medicine nodes to obtain vector representations corresponding to the medicine nodes;
the classification layer is configured to obtain, by using the vector representations of the medicine nodes in the medicine node pair, a classification result indicating whether the medicine node pair has synergism; and
a training objective of the model training unit is to minimize a difference between the classification result and the corresponding label.

13. The electronic device according to claim 12, wherein the preset neighborhood range comprises: a first-order protein neighbor node and neighbor protein nodes of the first-order protein neighbor node.

14. The electronic device according to claim 12, wherein edges between the medicine nodes in the relation graph are identified by cell lines, and the label of the training sample comprises cell line tags; and
the classification result is whether a medicine pair has synergism in the cell lines.

15. The electronic device according to claim 12, wherein, when performing graph convolution processing on the subgraphs corresponding to the medicine nodes, the graph convolutional network layer is specifically configured to:
embed features of the nodes and features of the edges comprised in the subgraphs corresponding to the medicine nodes; and
aggregate feature vectors of the nodes and feature vectors of the edges obtained by embedding, to obtain the vector representations corresponding to the medicine nodes.

16. The electronic device according to claim 15, wherein initial values of the feature vectors of the edges are pre-trained by a disease classification task; and
initial values of the feature vectors of the nodes are pre-obtained by a compoundcompound interaction (CCI) task.

17. The electronic device according to claim 11, wherein the medicine synergism prediction model comprises a graph convolutional network layer and a classification layer;
the graph convolutional network layer is configured to acquire, from the relation graph, subgraphs corresponding to the medicine nodes in the inputted medicine node pair, the subgraphs corresponding to the medicine nodes comprising medicine nodes, neighbor nodes of the medicine nodes within a preset neighborhood range and edges between the medicine nodes and the neighbor nodes; and performing graph convolution processing on the subgraphs corresponding to the medicine nodes to obtain vector representations corresponding to the medicine nodes;
the classification layer is configured to obtain, by using the vector representations of the medicine nodes in the medicine node pair, a classification result indicating whether the medicine node pair has synergism; and
a training objective of the model training unit is to minimize a difference between the classification result and the corresponding label.

18. A non-transitory computer readable storage medium with computer instructions stored thereon, wherein the computer instructions are used for causing a computer to perform a method for establishing a medicine synergism prediction model, wherein the method comprises:
acquiring a relation graph, nodes in the relation graph comprising medicine nodes and protein nodes, and edges indicating that interaction exists between the nodes;
collecting, from the relation graph, a medicine node pair with definite synergism and a label of whether the medicine node pair has synergism as training samples; and
training the medicine synergism prediction model by taking the medicine node pair in the training samples as input to the medicine synergism prediction model and taking the label of whether the medicine node pair has synergism as target output; wherein the medicine synergism prediction model is obtained by learning the relation graph based on a graph convolutional network.

19. The non-transitory computer readable storage medium according to claim 18, wherein the medicine synergism prediction model is obtained by learning the subgraph formed by some nodes and edges in the relation graph based on the graph convolutional network.

20. The non-transitory computer readable storage medium according to claim 18, wherein the medicine synergism prediction model comprises a graph convolutional network layer and a classification layer;
the graph convolutional network layer is configured to acquire, from the relation graph, subgraphs corresponding to the medicine nodes in the inputted medicine node pair, the subgraphs corresponding to the medicine nodes comprising medicine nodes, neighbor nodes of the medicine nodes within a preset neighborhood range and edges between the medicine nodes and the neighbor nodes; and performing graph convolution processing on the subgraphs corresponding to the medicine nodes to obtain vector representations corresponding to the medicine nodes;
the classification layer is configured to obtain, by using the vector representations of the medicine nodes in the medicine node pair, a classification result indicating whether the medicine node pair has synergism; and
a training objective of the medicine synergism prediction model is to minimize a difference between the classification result and the corresponding label.
